(19) United States
(12) Patent Application Publication
(10) Pub. No.: US 2016/0374911 A1
(43) Pub. Date: Dec. 29, 2016

(54) PLANT EXTRACT COMPOSITION FOR SKIN WHITENING AND REDUCING MELANIN AS WELL AS APPLICATION THEREOF

(71) Applicant: CALIWAY BIOPHARMACEUTICALS CO., LTD., Taipei City (TW)

(72) Inventor: Yu-Fang Ling, Taipei City (TW)

(21) Appl. No.: 15/191,899

(22) Filed: Jun. 24, 2016

(30) Foreign Application Priority Data
Jun. 29, 2015 (TW) 104120911

(51) Int. Cl. A61K 8/35; A61Q 19/02; A61K 8/97; A61K 8/34

(52) U.S. Cl. CPC A61K 8/35; A61K 8/347; A61Q 19/02; A61K 8/97; A61K 2800/782

(57) ABSTRACT

Provided are a plant extract composition and a pharmaceutical composition for inhibiting tyrosinase activity, preventing melanogenesis or treating melanogenesis, wherein the plant extract composition contains curcumin and resveratrol by weight ratio from 4:1 to 1:4. The plant extract composition and the pharmaceutical composition are useful for preventing and treating pigmentation for skin whitening, and achieve the effect of whitening skin and reducing pigmentation for a subject in topical site.

### CROSS REFERENCE

This application claims priority under 35 U.S.C. §119(a) to Taiwan Patent Application No. 104120911, filed on Jun. 29, 2015, the content of which is hereby incorporated by reference in its entirety.

### BACKGROUND OF THE INVENTION

1. Field of the Invention

The present invention also relates to a pharmaceutical composition, and more particularly to a pharmaceutical composition comprising a plant extract composition and suitable pharmaceutically acceptable excipient and more particularly to a plant extract composition for skin whitening, preventing pigmentation, or treating pigmentation, wherein the plant extract composition comprises a specific ratio of curcumin and resveratrol. The present invention also relates to a method of the pharmaceutical composition for skin whitening, preventing pigmentation, or treating pigmentation.

2. Description of the Prior Arts

Skin color is dominantly determined by melanin contents. Generally, melanin in the skin plays the role of natural protective barrier and resists ultraviolet radiation to prevent photocarcinogenesis. Excess melanin resulted in hyperpigmentation phenomena such as age spots, freckles, dark spots, liver spots, or wound healing process cause displeasure. Dermal melanin, which is produced by melanocytes, is transported to keratinocytes in the melanosome organelle by dendritic structure. The numbers of dermal melanocytes among human races are similar, however, number of melanosome and extent of melanin in keratinocytes determine the skin color.

Melanogenesis process is induced by UV exposure or melanocyte-stimulating hormone (α-MSH), which is secreted by keratinocytes to promote melanin synthesis. The biosynthetic pathway of melanin is a multi-steps process involving tyrosinase, tyrosinase related protein 1 (TRP-1), and tyrosinase related protein 2 (TRP-2, also called Dopachrome tautomerase, DCT) to convert tyrosine sequentially to L-DOPA, L-dopaquinone, dopachrome, 5,6-dihydroxyindole, indole-5,6-quinone, and melanin. Tyrosinase is the key enzyme for melanin synthesis and the regulation of tyrosinase activity was widely studied in reducing pigmentation and skin whitening In addition, post-inflammatory hyperpigmentation (PIH) caused by injury is another pathway of melanogenesis. Generally, PIH can be categorized as epidermal PIH and dermal PIH. When inflammation occurs in the epidermis, arachidonic acid is metabolized to prostaglandins E2 (PGE2) and leukotriene (LTC4) by cyclooxygenase and lipoxygenase respectively. After stimulation by inflammatory factors, melanocytes will facilitate melanogenesis as well as the melanin transfer to the surrounding keratinocytes. On the other hand, with basal cell layer in the junction of epidermis and dermis in inflammation, a large quantity of melanosome will be swallowed by melanophages, then resulting in deeper dark brown or blue-gray precipitation. PIH may be caused by acne, chicken pox, zoster, dermatitis or any skin inflammation due to wounds. Whitening ingredients used currently to inhibit melanogenesis or pigmentation include magnesium ascorbyl phosphate, sodium ascorbyl phosphate, ascorbyl glucoside, kojic acid, arbutin, ellagic acid, chamomile ET, 5,5′-dipropyl-biphenyl-2,2′-diol, tranexamic acid, potassium methoxysalicylate and hydroquinone.

Metal salts and glycated derivatives of vitamin C are used to inhibit melanogenesis by antioxidation via reducing L-dopaquinone. Vitamin C is considered to be very safe and able to prevent the formation of free radicals. However, it is unstable when oxidization occurs. Therefore, the stability of vitamin C can be enhanced through the binding with metal ion or glycation. Such derivatives include magnesium ascorbyl phosphate, sodium ascorbyl phosphate or ascorbyl glucoside. Since tyrosinase is an oxidase with divalent copper ion in the active center, a structure for inhibiting tyrosinase and then blocking melanogenesis should be able to combine or compete with copper ions, such as kojic acid. Kojic acid could combine with copper ion of tyrosinase to reduce the activity of tyrosinase hence reducing the conversion of tyrosine to L-DOPA and L-dopaquinone. Materials such as arbutin can compete with tyrosine to reduce the substrate for tyrosinase. In addition, hydroquinone was used to be cytotoxic agent to melanocyte by free radicals, and improper use of hydroquinone would lead to skin irritation, dermatitis, abnormal pigmentation, PIH and other side effects. Therefore, hydroquinone is classified as medicinal ingredients and is forbidden from use as an ingredient in cosmetics; furthermore, the content of hydroquinone in medicine could not exceed 5%.

The ingredients used in skin whitening through different mechanisms, such as blocking tyrosinase to reduce melanogenesis, blocking melanosome transferred from melanocytes to keratinocytes, inhibiting tyrosinase activity, promoting the metabolism of melanin in keratinocytes, or blocking UV rays. Vitamin C, tranexamic acid or vitamin B are attempted to treat speckle or whiten skin by intravenous injection, but this mode of administration is not legal in any country and no obvious whitening effects were observed, moreover, it is thought to increase the risk of allergies. In addition, if the products of administration are not sterilized completely, there will be a high risk of phlebitis, cellulitis, sepsis and other serious side effects.

Researches and development on whitening-related products to meet the market need have never ceased, and the consumer's demand for speckle treatment and skin whitening remains constant. However, ingredients currently used in whitening or removal of melanin still have room for improvement. Therefore, better safety and efficacy of melanin inhibition is eagerly required regarding medical cosmetics on the market.

Besides, in whitening ingredient or product development related fields, different drugs are administrated prior to α-MSH stimulation in a lot of experiments, and then the inhibitory effects on melanin within the groups are compared; or some ingredients are conducted only in vitro, and then directly mixed with drugs and tyrosine to compare the inhibitory effect on tyrosinase activity. As the reaction sequences of melanogenesis in vivo and the above-said approach are different, even if the result of the above-said approach is good, the whitening effect in real application still falls behind expectation.

### SUMMARY OF THE INVENTION

The objective of the present invention is to provide a composition for use in skin whitening, preventing pigmentation, or treating pigmentation, even in the case of melanogenesis mode, comprising: curcumin and resveratrol, for inhibiting tyrosinase significantly.

The present invention provides a plant extract composition for skin whitening, preventing pigmentation, or treating pigmentation, wherein a weight ratio of curcumin to resveratrol is from 4:1 to 1:4.

Preferably, the weight ratio of curcumin to resveratrol is from 4:1 to 3:2.

More preferably, the weight ratio of curcumin to resveratrol is 4:1.

According to the present invention, the term “curcumin” as used herein includes any source, is not limited to turmeric extract. Preferably, the concentration of the curcumin used in the present invention is from 95% to 100%.

The present invention further provides a pharmaceutical composition for skin whitening, preventing pigmentation or treating pigmentation, wherein the pharmaceutical composition comprises a therapeutically effective amount of the above plant extract composition and a pharmaceutically acceptable excipient, wherein the pharmaceutically acceptable excipient is applicable for injection, implantation and external formulation.

According to the present invention, the term “therapeutically effective amount” as used herein, refers to a dosage to inhibit tyrosinase activity and melanogenesis. The therapeutically effective amount for inhibiting tyrosinase activity and melanogenesis is determined by administering the pharmaceutical composition in an effective amount, and measuring the inhibitory effect on melanin and the inhibitory effect on tyrosinase activity in a specific period.

According to the present invention, the pharmaceutical composition of the present invention could be available by conventional methods, the excipient includes, but is not limited to, binders, disintegrating agents, dispersing agents, fillers, stabilizers, diluents and dyes. Preferably, the term “pharmaceutically acceptable excipient” as used herein includes any physiologically compatible and all solvents, dispersion medium, antibacterial and antifungal agents, isotonic and absorption delaying agents and analogues thereof. For example, the pharmaceutically acceptable excipients include one or more combination of water, saline, phosphate buffered saline (PBS), dextrose, glycerol, ethanol and its analogues. Preferable combination includes isotonic agents, for example, sugar or polyol such as mannitol, sorbitol, or sodium chloride. The pharmaceutically acceptable excipients further include microscale auxiliary substances such as wetting or emulsifying agents, preservatives or buffers.

In accordance with the present invention, the pharmaceutical composition for skin whitening, preventing pigmentation or treating pigmentation is prepared for multiple forms, including, but not limited to, liquid, semi-solid and solid dosage, such as liquid solution (including injectable and infusible solution), dispersions or suspensions, tablets, pills, powders, liposomes and suppositories. Preferred form depends on the mode of administration and therapeutic application of expectations. In an embodiment of the present invention, the pharmaceutical composition at the effective amount is oral, by infusion solutions or by topical external formulation. Preferably, the pharmaceutical composition of the present invention is administered in the form of subcutaneous injection or intravenous injection. More preferably, the external formulation includes, but is not limited to, emulsion, gel, ointment, cream, patch, liniment, powder, aerosols, spray, lotion, serum, paste, foam, drop, suspension and salve.

More preferably, the pharmaceutical composition of the present invention used as an external formulation further comprises additives, wherein the additives include, but are not limited to, water, alcohols, glycol, hydrocarbons (such as petroleum jelly and white petrolatum), wax (such as paraffin and yellow wax), preserving agents, antioxidants, surfactants, absorption enhancers, stabilizing agents, gelling agents (such as microcrystalline cellulose and carboxymethylcellulose), active agents, humectants, odor absorbers, fragrances, pH adjusting agents, chelating agents, emulsifiers, occlusive agents, emollients, thickeners, solubilizing agents, penetration enhancers, anti-irritants, colorants and propellants. The species and the amount of additive could be selected by a skilled person in the art.

The plant extract composition and the pharmaceutical composition of the present invention could be formulated to skin care or cosmetic products for external application by adding vehicles. Preferably, the vehicles include, but are not limited to, emulsions (such as water-in-oil or oil-in-water emulsion), cream, lotion, solution (such as an aqueous solution or water-alcohol solution), anhydrous base (such as lipstick or powder), foam, gel, cream, mask, sprays and ointments.

Preferably, the pharmaceutically acceptable excipient is nonionic surfactant. More preferably, the nonionic surfactant includes, but is not limited to, polyoxyethylene castor oil derivatives, alkanolamides, polyethylene oxide, Span or Tween.

More preferably, the polyoxyethylene castor oil derivatives include, but are not limited to Cremophor® ELP, Cremophor® EL or Cremophor RH® 40.

More preferably, the alkanolamides includes, but is not limited to, cocamide monoethanolamine (cocamide MEA) or coconut diethanolamide (cocamide DEA).

More preferably, the polyethylene oxide includes, but is not limited to, laureth-4, ceteth-2, ceteth-20, steareth-2, PEG-8 laurate, PEG-8 stearate, PEG-8 oleate, PEG-8 dioleate, PEG-40 stearate. PEG-100 stearate or PEG-150 distearate.

More preferably, Span or Tween include, but are not limited to, sorbitan monolaurate, sorbitan monopalmitate, sorbitan monostearate, sorbitan tristearate, sorbitan monooleate, POE (20) sorbitan monolaurate, POE (20) sorbitan monopalmitate, POE (20) sorbitan monostearate, POE (20) sorbitan tristearate, POE (20) sorbitan monooleate, POE (20) sorbitan trioleate. Generally, the HLB value of the nonionic surfactant is from about 8 to about 18, such as POE (20) oleyl alcohol, polyoxyethylene stearyl alcohol, polyoxyethylene monostearate, polyoxyethylene sodium oleate, or polyoxyethylene lauryl ether.

The present invention further provides a method for skin whitening, preventing pigmentation or treating pigmentation comprising a step of administering to a subject in need thereof a therapeutically effective amount of a pharmaceutical composition in the effective amount containing a pharmaceutically acceptable excipient.

Preferably, the subject includes animal Generally, the subject is human.

Preferably, the administration of the pharmaceutical composition includes, but is not limited to local injection, intravenous injection, implantation or external application.

Preferably, the therapeutically effective amount of the pharmaceutical composition by implantation is from 0.05 mg/cm² and 50 mg/cm².

Preferably, the therapeutically effective amount of the pharmaceutical composition by local injection or intravenous injection is between from 0.005 mg/cm² to 5 mg/cm².

Preferably, the therapeutically effective amount of the pharmaceutical composition by external application is from 0.01% to 10%.

The plant extract composition and the pharmaceutical composition of the present invention can be used to lighten skin color concomitant with preventing or treating hyperpigmentation, wherein hyperpigmentation includes, but is not limited to, age spots, liver spots, freckles, hyper pigmentation caused by inflammation or trauma, or hyperpigmentation after sun exposure. The plant extracts composition of the present invention has high safety without cytotoxicity on cell viability assay. The plant extract composition of the present invention is administrated under α-MSH stimulated melanogenesis and the amount of melanin is reduced effectively. In the present invention, a stimulated melanogenesis mode is used to show that plant extract composition reduced melanin content to whiten skin.

### BRIEF DESCRIPTION OF THE DRAWINGS

FIG. 1 shows the effect of control group (DMEM), arbutin, resveratrol, curcumin and the compositions AW-001-C2, AW-001-C3, AW-001-C5 or AW-001-C7 of the present invention on cell viability of mouse melanoma B16-F10 cells.

FIG. 2A shows the effect of arbutin, resveratrol, curcumin, compositions AW-001-C2, AW-001-C3, AW-001-C5, or AW-001-C7 of the present invention compared to that of control group (α-MSH) on melanogenesis rates of mouse melanoma B16-F10 cells.

FIG. 2B shows the effect of arbutin, resveratrol, curcumin and the compositions of AW-001-C2, AW-001-C3, AW-001-C5 or AW-001-C7 of the present invention on melanin inhibition in mouse melanoma B 16-F10 cells.

FIG. 3A shows the effect of arbutin, resveratrol, curcumin and the compositions of AW-001-C2, AW-001-C3, AW-001-C5 or AW-001-C7 of the present invention on tyrosinase activity in mouse melanoma B 16-F10 cells.

FIG. 3B shows the effect of arbutin, resveratrol, curcumin and the compositions of AW-001-C2, AW-001-C3, AW-001-C5 or AW-001-C7 of the present invention on tyrosinase inhibition in mouse melanoma B16-F10 cells.

FIG. 4 shows the cell viability of the formulations of different excipients such as distilled deionized water (as DDW control group or AW-001-C2-DDW group), mannitol (as mannitol control group or AW-001-C2 mannitol group) for oral formulation, and Cremophor® ELP (as Cremophor® ELP control group or AW-001-C2 Cremophor® ELP group) for injection or external formulation with/without AW-001-C2.

FIG. 5 shows the melanogenesis of the formulations of different excipients such as distilled deionized water, mannitol, and Cremophor® ELP with/without AW-001-C2 under microscope observation, wherein arrowheads refer to formulation precipitation. (left column: from top to bottom was DDW control group, mannitol control group and Cremophor® ELP control group; right column: from top to bottom was AW-001-C2-DDW group, AW-001-C2-mannitol group and AW-001-C2 Cremophor® ELP group)

FIG. 6 shows the melanogenesis of the formulations of different excipients such as distilled deionized water, mannitol, and Cremophor® ELP with/without AW-001-C2.

### DETAILED DESCRIPTION OF THE PREFERRED EMBODIMENTS

Other objectives, advantages and novel features of the invention will become more apparent from the following detailed description when taken in conjunction with the accompanying drawings.

The weight ratio of the components of the plant exact compositions of the present invention:

The composition AW-001-C2 comprises curcumin and resveratrol, wherein the weight ratio of curcumin to resveratrol is 4:1.

The composition AW-001-C3 comprises curcumin and resveratrol, wherein the weight ratio of curcumin to resveratrol is 1:4.

The composition AW-001-C5 comprises curcumin, resveratrol, and quercetin, wherein the weight ratio of curcumin to resveratrol to quercetin is 2:0:3.

The composition AW-001-C7 comprises curcumin and resveratrol, wherein the weight ratio of curcumin to resveratrol is 3:2.

### Example 1

### Cell Viability Assay

The purpose of the experiment is to compare single plant extract and the compositions of the present invention for cell viability. Mouse melanoma B16-F10 cells were used to examine the cell viability, and divided into eight groups as control group (DMSO), arbutin, resveratrol, curcumin, and compositions AW-001-C2, AW-001-C3, AW-001-C5, or AW-001-C7 of the present invention. All groups were repeatedly tested for 3 times, and the cell viabilities were analyzed by flow cytometer.

1×10⁵ B16-F10 cells were cultured in 6-well plates. After incubation for 24 hours, except for control group (treated with DMSO), the other groups were then treated respectively with 250 ppm arbutin, 6 ppm resveratrol, 8 ppm curcumin, 8 ppm AW-001-C2, 8 ppm AW-001-C3, 8 ppm AW-001-C5, or 8 ppm AW-001-C7 for 48 hours. Trypsin-EDTA was then added for cell collection and 5×10⁵ cells per group were transferred into flow tubes. After centrifugation, cells were washed twice by PBS and dyed by propidium iodide (PI) for analyzing the expression of PI.

Accordingly, the term “ppm” used herein refers to one liter (L) containing one microliter (μl) substance, or to one liter (L) containing one milligram (mg) substance. In this example, example 2, and example 3, arbutin was dissolved in DMSO to form 250,000 ppm, and diluted into 250 ppm via culture medium while in use. In this example, example 2, and example 3, the compositions of the present invention were dissolved in DMSO respectively to form 8,000 ppm, diluted into 8 ppm via culture medium while in use, and so on.

Generally, as known in pharmacological field, when the concentration of drug during cell experiments was 8 ppm, the dose of administration for human was 8 times to 200 times, i.e., the administration for human was from 64 ppm to 1600 ppm. Preferably, the administration for human was from 50 times to 200 times, i.e., the administration for human was from 400 ppm to 800 ppm.

Generally, as known in pharmacological field, when the effective concentration of skin-whitening drug used in cell experiments was 8 ppm, the therapeutically effective amount of the skin-whitening drug by local injection or intravenous injection is from 0.005 mg/cm² to 5 mg/cm².

Generally, as known in pharmacological field, when the effective concentration of skin-whitening drug used in during cell experiments was 8 ppm, the therapeutically effective amount of the skin-whitening drug by implantation is from 0.05 mg/cm² to 50 mg/cm².

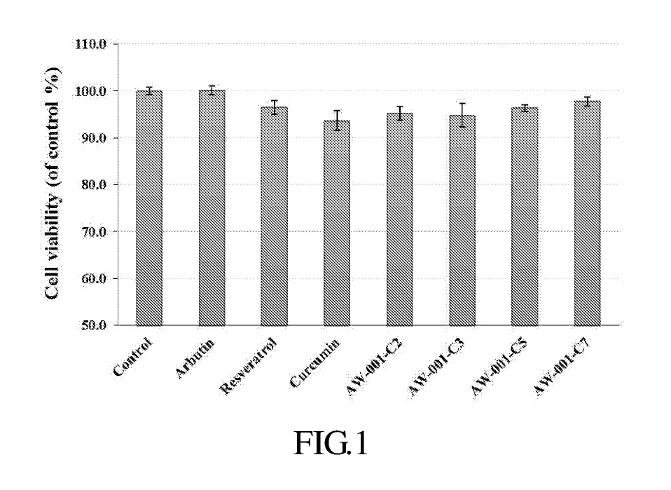

As shown in FIG. 1, the result indicated that cell viability was not affected in either single plant extract or compositions AW-001-C2, AW-001-C3, AW-001-C5, or AW-001-C7 of the present invention compared with that of control group. All data represent no statistical difference among the groups, which means that single plant extract or compositions AW-001-C2, AW-001-C3, AW-001-C5, or AW-001-C7 of the present invention do not cause melanoma cells death.

### Example 2

### Inhibition of Melanogenesis (Melanogenesis Induction Firstly, and Then Administration)

The purpose of the experiment is to compare single plant extract and the compositions of the present invention for inhibiting melanogenesis. Mouse melanoma B16-F10 cells were used to examine the inhibition of melanogenesis, and divided into eight groups as control group (α-MSH), arbutin, resveratrol, curcumin, and compositions AW-001-C2, AW-001-C3, AW-001-C5, or AW-001-C7 of the present invention. All groups were repeatedly tested for 3 times, and the amounts of melanin were analyzed.

2×10⁵ B16-F10 cells were cultured in 6-well plates. After incubation for 24 hours, 10 ng/ml α-MSH was respectively added for 30 minutes. Except for control group, the other groups were then treated respectively with 250 ppm arbutin, 6 ppm resveratrol, 8 ppm curcumin, 8 ppm AW-001-C2, 8ppm AW-001-C3, 8 ppm AW-001-C5, or 8 ppm AW-001-C7 for 48 hours. Then, after centrifuged at 120 g for 5 minutes at 20° C., the cells were collected and lysed in 1N NaOH solution containing 10% DMSO to form samples. The samples were mixed and heated at 80° C. for another 1.5 hours. After the heating, samples were cooled, and absorbance at a wavelength of 475 nm was measured using microplate reader (SpectraMax® M2e Multimode Microplate Reader) to calculate melanin inhibition.

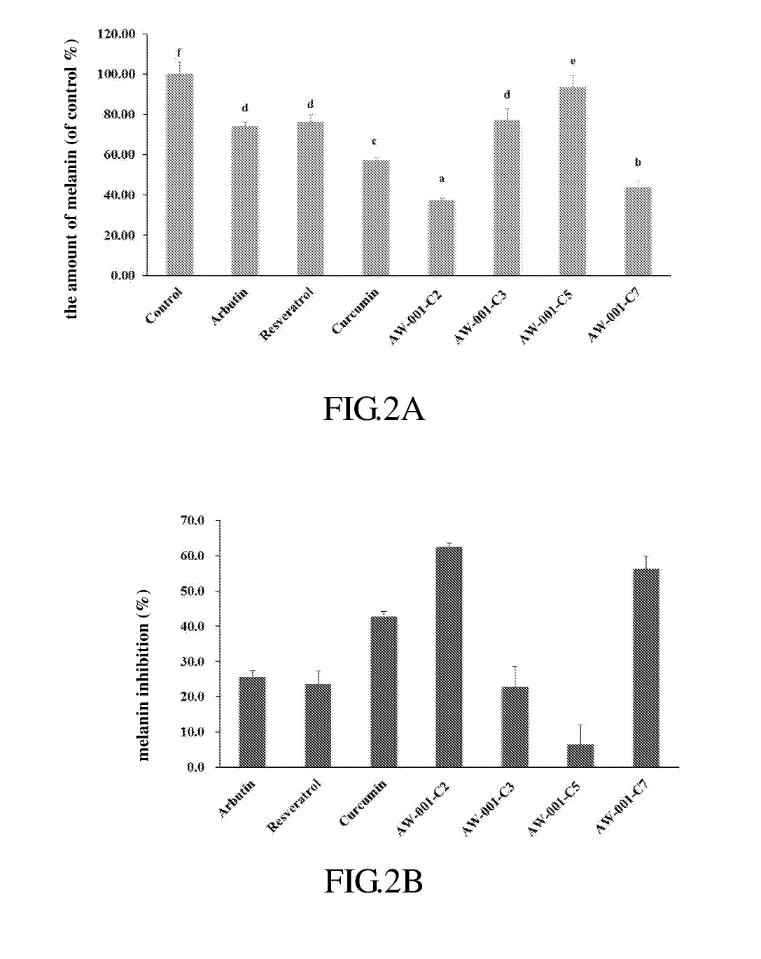

As shown in FIG. 2, melanin in each group was inhibited. Even though single plant extract could inhibit melanogenesis, compositions of AW-001-C2 or AW-001-C7 of the present invention can inhibit melanogenesis significantly compared with that in the single plant extract such as resveratrol, curcumin, or arbutin. The compositions AW-001-C2, AW-001-C3, or AW-001-C7 of the present invention can also inhibit melanogenesis effectively. Compared with that of control group, the inhibitory effect on melanin of the formulations AW-001-C2, AW-001-C3, or AW-001-C7 was 63.5%, 24.2% and 56.1%, respectively, while no melanin inhibition was found in the composition AW-001-C5, compared with that of control group. The compositions AW-001-C2 and AW-001-C7 exerted best melanin inhibitory effects and even better than that of 250 ppm arbutin group. After calibration, the melanin inhibitory effect of compositions AW-001-C2 and AW-001-C7 is 76.2 and 68.7 folds respectively better than that of arbutin group.

### Example 3

### Inhibition of Tyrosinase (Melanogenesis Induction Firstly, and Then Administration)

The purpose of the experiment is to analyze the inhibition of tyrosinase activity with the treatments of single plant extract or the compositions of the present invention by measuring the amount of dopaquinone conversed from L-DOPA. Mouse melanoma B16-F10 cells were used and arranged to eight groups including control group (α-MSH), arbutin, resveratrol, curcumin, compositions AW-001-C2, AW-001-C3, AW-001-C5, or AW-001-C7 of the present invention.

2×10⁵ B16-F10 cells were cultured in 6-well plates. After incubation for 24 hours, 10 ng/ml α-MSH were respectively added for 30 minutes incubation. Except for control group, the other groups were then treated respectively with 250 ppm arbutin, 6 ppm resveratrol, 8 ppm curcumin, 8 ppm AW-001-C2, AW-001-C3, AW-001-C5, or AW-001-C7 for 48 hours. Trypsin-EDTA was then added for cell collection and cells were washed by PBS. Tyrosinase protein was extracted and quantified with 0.1M PBS containing 1% Triton X-100 and 0.1 mM phenylmethanesulfonylfluoride (PMSF). 30 μg total protein was mixed with 0.4 mg/ml L-DOPA and the absorbance was measured at 405 nm wavelength every 10 minutes within one hour.

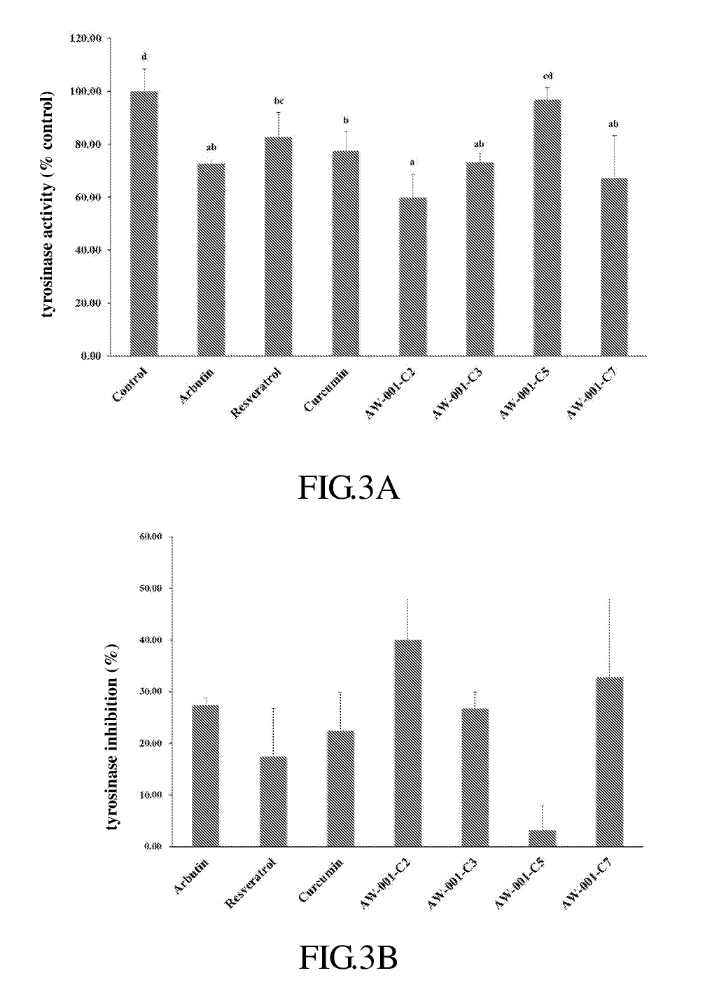

As shown in FIG. 3, the result indicated that arbutin, resveratrol, curcumin, compositions AW-001-C2, AW-001-C3 or AW-001-C7 of the present invention reduced the activity of tyrosinase within 60 minutes compared with that of control group. The inhibitory effects of the compositions of the present invention were better than that of single plant extract resveratrol, curcumin, or arbutin. Compared with that of control group, the inhibitory effect on tyrosinase of the compositions AW-001-C2, AW-001-C3, or AW-001-C7 was 42.2%, 24.7%, and 39.1%, respectively, while no inhibitory effect was found in formulation AW-001-C5 group. The compositions AW-001-C2 and AW-001-C7 exerted the best tyrosinase inhibition. After calibration, the compositions AW-001-C2 and AW-001-C7 compared with that of arbutin group, the tyrosinase inhibitory effect of compositions AW-001-C2 and AW-001-C7 was 45.6 folds and 37.4 folds respectively. The result is consistent to Example 2.

### Example 4

### Effect of Pharmaceutical Composition with Different Excipients

Due to DMSO is not a pharmaceutically acceptable solvent, the purpose of the experiment is to estimate the effects of formulations AW-001-C2 with different pharmaceutically acceptable excipients instead of with DMSO.

TABLE 1
Formulations AW-001-C2 with different
excipients and final concentration.

| | | | | Final |
|---|---|---|---|---|
| Sample | Group | Excipient | AW-001-C2 | concentration |
| 1 | Control | Distilled | − | — |
| | group | deionized water | | |
| 2 | | Distilled | + | 8 ppm |
| | | deionized water | | |
| 3 | Oral | Mannitol | − | — |
| 4 | formulation | | + | 8 ppm |
| 5 | Injection or | Cremophor ® | − | — |
| 6 | external | ELP | + | 8 ppm |
| | formulation | | | |

As shown in Table 1, sample 1 was distilled deionized water with no AW-001-C2 (as DDW control group).

Sample 2 was distilled deionized water with AW-001-C2 (as AW-001-C2-DDW group), and obtained from the following step: weighing 16.0 mg curcumin mg and 4.0 mg resveratrol respectively into 15 ml centrifuge tube; adding injectable saline to 5 g (total weight) to the centrifuge tube and mixing to obtain 4000 ppm (4 mg/g) AW-001-C2 aqueous solution, and AW-001-C2 aqueous solution was diluted with 500-fold culture medium to form final concentration 8 ppm AW-001-C2 aqueous solution while in use.

Sample 3 was mannitol with no AW-001-C2 (as mannitol control group), obtained from the following step: weighing 1.5 g mannitol into 15 ml centrifuge tube; adding saline to 10 g (total weight)to the centrifuge tube and mixing for 10 minutes to obtain 15 wt % mannitol solution.

Sample 4 was mannitol with AW-001C2 (as AW-001-C2 mannitol group), obtained from following step: weighing 16.0 mg curcumin and 4.0 mg resveratrol respectively into 15 ml centrifuge tube; adding 15 wt % mannitol solution to the centrifuge tube to 5 g (total weight) and mixing to obtain 4000 ppm (4 mg/g) AW-001-C2 mannitol solution, and AW-001-C2 mannitol solution was diluted with 500-fold culture medium to form final concentration 8 ppm AW-001-C2 mannitol solution while in use (the final concentration of mannitol was 0.03%).

Sample 5 was Cremophor® ELP with no AW-001-C2 (as Cremophor® ELP control group), obtained from the following step: weighing 3.0 g Cremophor® ELP into 50 ml beaker; adding saline to the beaker to 20 g (total weight) and stirring for 3 hours to obtain 15 wt % Cremophor® ELP solution. Sample 6 was Cremophor® ELP with AW-001C2 (as AW-001-C2 Cremophor® ELP group), obtained from the following step: weighing 64.0 mg curcumin and 16.0 mg resveratrol respectively into 50 ml beaker; adding and stirring 16 ml dichloromethane (DCM) to the 50 ml beaker for complete dissolution; adding and mixing 3 g Cremophor® ELP to the 50 ml beaker for 10 minutes to 20 minutes, and then evaporated DCM for 2 hours to 5 hours; adding saline to 20 g (total weight) and stirring for 2 hours to 4 hours at 100 rpm to 300rpm to obtain 4000 ppm (4 mg/g) AW-001-C2 Cremophor® ELP solution, and AW-001-C2 mannitol solution was diluted with 500-fold culture medium to form final concentration 8 ppm AW-001-C2 mannitol solution while in use (the final concentration of mannitol was 0.03%).

2×10⁵ B16-F10 cells were cultured in 6-well plates. After incubation for 24 hours, B16-F10 cells were treated by the samples for 3 hours as shown in Table 1 and 10 ng/ml α-MSH was then added respectively for another 48 hours incubation. Trypsin-EDTA was added for cell collection and stained with trypan blue for cell viability analysis in 3 independent experiments.

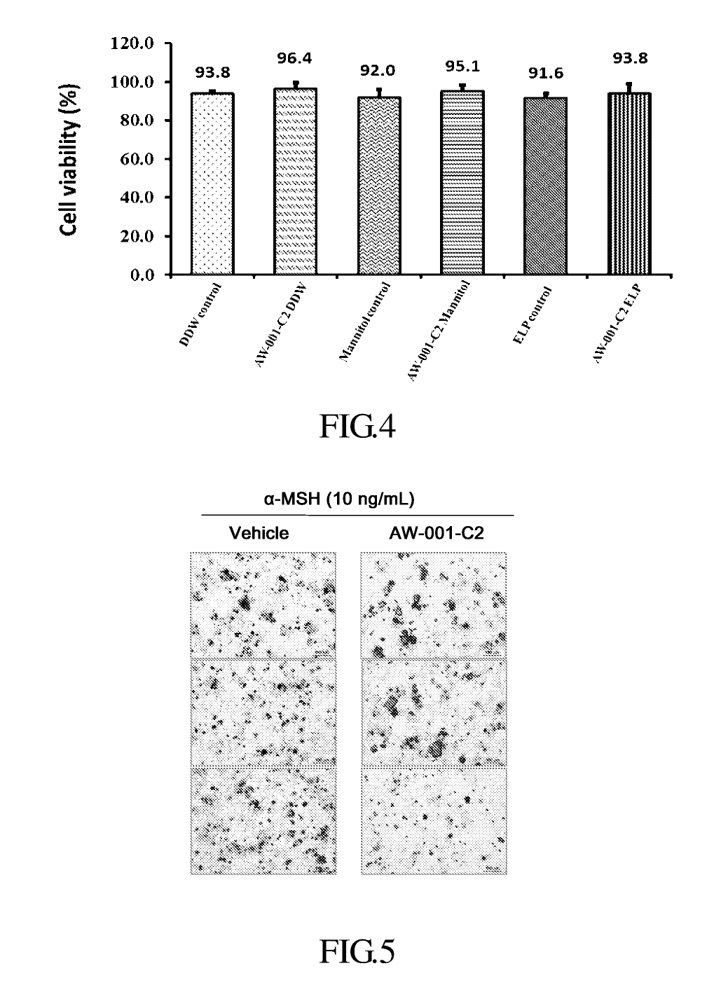

As shown in FIG. 4, the result indicated that cell viability was not affected compared with that of control group. No statistical difference among groups revealed that formulation AW-001-C2 with different excipients of the present invention affected melanoma cells in similar extent.

### Example 5

### Inhibition of Melanogenesis

2×10⁵ B16-F10 cells were cultured in 6-well plates. After incubation for 24 hours, B16-F10 cells were treated by the samples as shown in Table 1 respectively for 3 hours. Then, 10 ng/ml α-MSH was respectively added for 48 hours. Melanogenesis of the formulation AW-001-C2 with different excipients can be observed under microscope. After centrifuged at 120 g for 5 minutes under 20° C. to collect cells, the cells were redissolved by 1N NaOH (containing 10% DMSO) to form samples respectively. The samples were mixed and heated at 80° C. for 1.5 hours respectively. Until the samples were cooled, microplate reader (SpectraMax® M2e Multimode Microplate Reader) was used to measure absorbance at a wavelength of 475 nm and calculate melanin inhibition.

As shown in FIG. 5, compared with AW-001-C2 DDW control group and AW-001-C2 mannitol group, the melanogenesis of AW-001-C2 Cremophor® ELP group was significantly lower. The results showed that formulation AW-001-C2 with Cremophor® ELP excipient can effectively inhibit α-MSH-induced melanin production.

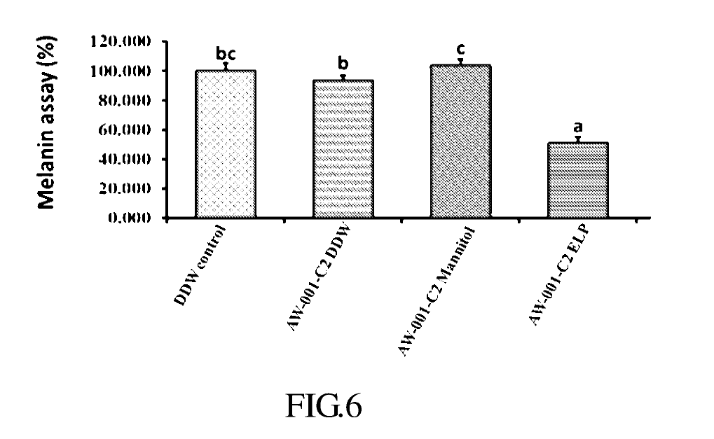

As shown in FIG. 6, compared with DDW control group, the melanogenesis of AW-001-C2 Cremophor® ELP group was lower than that of DDW control group for 49.24%. Compared with AW-001-C2 mannitol group, AW-001-C2 Cremophor® ELP group was significantly lower than that of AW-001-C2 mannitol group for 51.1%. This means the pharmaceutical composition comprising formulation AW-001-C2 with Cremophor® ELP can effectively inhibit melanogenesis.

What is claimed is:

1. A pharmaceutical composition for whitening skin, preventing pigmentation, or treating pigmentation, wherein the pharmaceutical composition comprises a therapeutically effective amount of a plant extract composition and a pharmaceutically acceptable excipient, wherein the plant extract composition comprises curcumin and resveratrol, and a weight ratio of curcumin to resveratrol is from 4:1 to 1:4; wherein the pharmaceutically acceptable excipient is applicable for injection, implantation and external formulation.

2. The pharmaceutical composition as claimed in claim 1, wherein the pharmaceutically acceptable excipient is nonionic surfactant.

3. The pharmaceutical composition as claimed in claim 2, wherein the nonionic surfactant comprises polyoxyethylene castor oil derivatives, alkanolamides, polyethylene oxide, Span or Tween.

4. The pharmaceutical composition as claimed in claim 3, wherein the polyoxyethylene castor oil derivatives is Cremophor® ELP, Cremophor® EL or Cremophor RH 40.

5. The pharmaceutical composition as claimed in claim 4, wherein the weight ratio of curcumin to resveratrol is from 4:1 to 3:2.

6. The pharmaceutical composition as claimed in claim 5, wherein the weight ratio of curcumin to resveratrol is 4:1.

7. A method for skin whitening, preventing pigmentation, or treating pigmentation, comprising a step of administering to a subject in need thereof a therapeutically effective amount of the pharmaceutical composition as claimed in claim 1.

8. The method as claimed in claim 7, wherein the pharmaceutically acceptable excipient is nonionic surfactant.

9. The method as claimed in claim 8, wherein the nonionic surfactant comprises polyoxyethylene castor oil derivatives, alkanolamides, polyethylene oxide, Span or Tween.

10. The method as claimed in claim 9, wherein the polyoxyethylene castor oil derivatives is Cremophor® ELP, Cremophor® EL or Cremophor® RH 40.

11. The method as claimed in claim 7, wherein the weight ratio of curcumin to resveratrol is from 4:1 to 3:2.

12. The method as claimed in claim 7, wherein the subject is an animal

13. The method as claimed in claim 12, wherein the subject is a human.

14. The method as claimed in claim 7, wherein the administration of the pharmaceutical composition comprises local injection, intravenous injection, implantation or external application.

15. The method as claimed in claim 14, wherein the therapeutically effective amount of the pharmaceutical composition by local injection or intravenous injection is from 0.005 mg/cm² to 5 mg/cm².

16. The method as claimed in claim 14, wherein the therapeutically effective amount of the pharmaceutical composition by implantation is from 0.05 mg/cm² to 50 mg/cm².

17. The method as claimed in claim 14, wherein the amount of the therapeutically effective amount of the pharmaceutical composition by external application is from 0.01% to 10%.
